(19) United States
(12) Patent Application Publication
(10) Pub. No.: US 2006/0155347 A1
(43) Pub. Date: Jul. 13, 2006

(54) Harmless wireless energy transmission to implant

(75) Inventor: Peter Forsell, Zug (CH)

Correspondence Address: NIXON & VANDERHYE, PC, 901 NORTH GLEBE ROAD, 11TH FLOOR, ARLINGTON, VA, 22203, US

(73) Assignee: POTENCIA MEDICAL AG

(21) Appl. No.: 10/527,989

(22) PCT Filed: Sep. 18, 2003
(86) PCT No.: PCT/SE03/01464

§ 371 Date: Oct. 13, 2005

(30) Foreign Application Priority Data
Sep. 20, 2002 (US) 60412014

(51) Int. Cl. A61N 1/08

(52) U.S. Cl. 607/61

(57) ABSTRACT

A transmission device (1) transmits an alternating magnetic field to a receiver (3) implanted in a human's body to supply energy drawn from the alternating magnetic field to an energy consuming implant in the human's body. The transmission device comprises a coil (6) adapted to generate the alternating magnetic field in a desired direction towards the implanted receiver. A shield (7) shields an operator's hand (2) from the alternating magnetic field generated by the coil. The shield includes a magnetizable core (8) extending in the coil (6) and a magnetizable casing (9) integrated with the core and surrounding the rear end of the coil and the circumference of the coil along at least a portion of the longitudinal extension of the coil.

The present invention relates to a transmission device and methods for transmission of an alternating magnetic field to a receiver implanted in a human's or animal's body to supply energy drawn from the alternating magnetic field to an energy consuming implant in the human's or animal's body.

Such a transmission device typically includes a coil that wirelessly co-operates with a coil of the implanted receiver. It has been found, however, that it is very unpleasant to a person to operate a handheld transmission device, because bad sensations occur in the person's hand while the transmission device is transmitting the alternating magnetic field. These bad sensations were not reduced significantly when the transmission device was encapsulated in a plastic case with a certain distance between the hand and the coil. Probably, the alternating magnetic field somehow unfavorably influences the nerves of the hand. It is not possible to shield the hand from the magnetic field by providing a steel shield between the hand and the coil, because such a steel shield would shorten the magnetic field. Because of the above-noted problems, prior hand-held transmission devices of coil-to-coil type have not been commercialized.

The object of the present invention is to provide an alternating magnetic field transmission device, which at least significantly reduces or even prevents unpleasant sensations to a person's hand during manual operation of the transmission device.

Another object of the invention is to provide an apparatus for wireless transfer of energy including a magnetic field transmission device, which at least significantly reduces or even prevents unpleasant sensations to a person's hand during manual operation of the transmission device.

Another object of the invention is to provide an apparatus for wireless transfer of energy including a magnetic field transmission device, which at least significantly reduces or even prevents environmental disturbances during operation of the transmission device.

Yet another object of the invention is to provide methods for harmless wireless transfer of energy to an energy consuming medical device implanted in a human's or animal's body.

Accordingly, in accordance with a first aspect of the present invention, there is provided a transmission device for transmitting an alternating magnetic field to a receiver, which is implanted in a human's or animal's body to supply energy drawn from the alternating magnetic field to an energy consuming implant in the human's or animal's body. The transmission device comprises a coil adapted to generate the alternating magnetic field in a desired direction towards the implanted receiver, the coil having a longitudinal extension, a front end to be directed towards the receiver and a rear end to be directed away from the receiver. The transmission device is characterized by a shield adapted to shield the environment, in particular an operator's hand holding the transmission device, from the alternating magnetic field generated by the coil except at the front end of the coil. The shield includes a magnetizable core extending in the coil and a magnetizable casing integrated with the core and surrounding the rear end of the coil and the circumference of the coil along at least a portion of the longitudinal extension of the coil.

The shield reduces the peaks or transients of the alternating magnetic field and the magnetic field itself in the direction towards, for example, an operator's hand while the transmission efficiency of the transmission device in the opposite direction, i.e. the direction towards the implanted receiver, is maintained. As a result, the unpleasant hand sensations previously experienced by operators testing prior hand-held transmission devices are practically eliminated by the transmission device of the invention. This improvement of the transmission device of the invention is of great significance, because the transmission device is intended for use daily by an operator, such as a doctor or nurse, to treat different patients with energy consuming implants, and a doctor or nurse would be very reluctant to use a transmission device that gives rise to the unpleasant hand sensations discussed above.

The casing may completely surround the coil except the front end thereof and the core may wholly extend along the longitudinal extension of the coil.

Alternatively, the casing may surround the circumference of the coil along a portion of the longitudinal extension of the coil and the core or/and coil may extend past the casing along the longitudinal extension of the coil, as seen in the direction towards the front end of the coil.

In accordance with an embodiment of the present invention, the casing includes a circular cylindrical wall and a circular gable wall joined to the cylindrical wall. The core extends centrally in the cylindrical wall from the gable wall and the coil is applied on the core with the rear end of the coil facing the gable wall. The cylindrical wall may be provided with cutouts, in order not to reduce the effective magnetic field too much. Alternatively, the cylindrical wall may be shorter than the core. For example, the cylindrical wall may extend axially from the gable wall along half the length of the core or a third of the length of the core. Depending on the strength of the transmitted magnetic field the length of the cylindrical wall may be more reduced or even eliminated. The coil may be shorter or longer than the core and/or the cylindrical wall.

In all embodiments of the invention, the shield preferably is made of ferrite or similar magnetizable material.

The transmission device suitably includes a plastic box, in which the coil and shield are arranged such that they are located at a distance, in the order of centimeters, from the operator's hand, when the operator holds the transmission device.

In accordance with a second aspect of the present invention, there is provided an apparatus for wireless transfer of energy from outside a human's or animal's body to an energy consuming medical device implanted in the human's or animal's body. The apparatus comprises a transmission device operable from outside the human's or animal's body for transmission of an alternating magnetic field, and a receiver implantable in the human's or animal's body for receiving and for drawing energy from the alternating magnetic field to be supplied to the energy consuming implanted medical device. The transmission device includes at least one coil for generating the alternating magnetic field in a desired direction towards the receiver, the coil having a longitudinal extension, a front end to be directed towards the receiver and a rear to be directed away from the receiver. The apparatus is characterized by at least one shield adapted to shield the environment from the alternating magnetic field generated by the coil except at the front end of the coil. The shield includes a magnetizable core extending in the coil and a magnetizable casing integrated with the core and surrounding the rear end of the coil and the circumference of the coil along at least a portion of the longitudinal extension of the coil.

The transmission device of the apparatus may be designed in accordance with the embodiments described above in connection with the first aspect of the invention.

Alternatively, the transmission device may include two identical transmitters that are to be placed at different sides of the implanted receiver, for example on the human's abdomen and back. The two transmitters may also be placed on a stand relative to the human's or animal's body so that the two transmitters are in the desired positions for transmitting the alternating magnetic fields towards the implanted receiver.

The apparatus may be used for supplying energy to implanted medical devices, such as adjustable restriction devices for treating obesity, heartburn and reflux disease, urinary and anal incontinence, infusion pumps, muscle stimulators, impotence or other wireless transfer of energy to implants.

In accordance with a third aspect of the present invention, the invention also provides a method for harmless wireless transfer of energy to an energy consuming medical device implanted in a human's or animal's body. The method comprises:

implanting in the human or animal a receiver capable of receiving and drawing energy from an alternating magnetic field to be supplied to the energy consuming medical device;

manually holding external to the body a transmission device capable of transmitting the alternating magnetic field, the transmission device including a coil having a longitudinal extension, a front end directed away from the hand holding the transmission device and a rear end facing the hand holding the transmission device; and

transmitting by means of the transmission device the alternating magnetic field to the implanted receiver. The method is characterized by shielding by means of a shield the hand holding the transmission device from the alternating magnetic field generated by the coil. The shield includes a magnetizable core extending in the coil of the transmission device and a magnetizable casing integrated with the core of the transmission device and surrounding the rear end of the coil and the circumference of the coil along at least a portion of the longitudinal extension of the coil.

In a more general aspect, the present invention also provides another method for harmless wireless transfer of energy to an energy consuming medical device implanted in a human's or animal's body, comprising:

implanting in the human or animal a receiver capable of receiving and drawing energy from an alternating magnetic field to be supplied to the energy consuming medical device;

providing an external transmission device capable of transmitting the alternating magnetic field and including a coil having a longitudinal extension, a front end and a rear end;

positioning the transmission device relative to the body so that the front end of the coil is directed towards the receiver and the rear end of the coil is directed away from the receiver; and

transmitting by means of the transmission device the alternating magnetic field to the implanted receiver. The method is characterized by shielding by means of a shield the environment from the alternating magnetic field generated by the coil except at the front end of the coil. The shield includes a magnetizable core extending in the coil of the transmission device and a magnetizable casing integrated with the core of the transmission device and surrounding the rear end of the coil and the circumference of the coil along at least a portion of the longitudinal extension of the coil.

The present invention is described in more detail in the following with reference to the accompanying drawings, in which

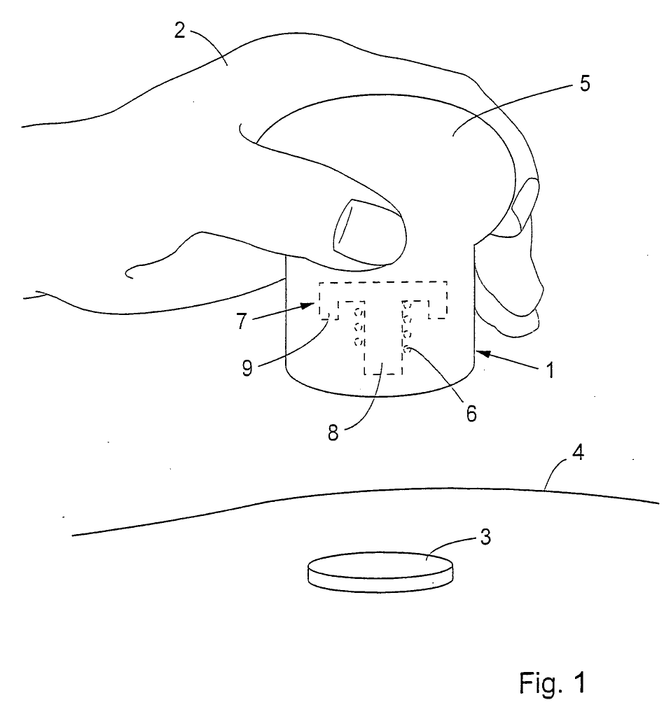

FIG. 1 schematically illustrates an apparatus of the present invention,

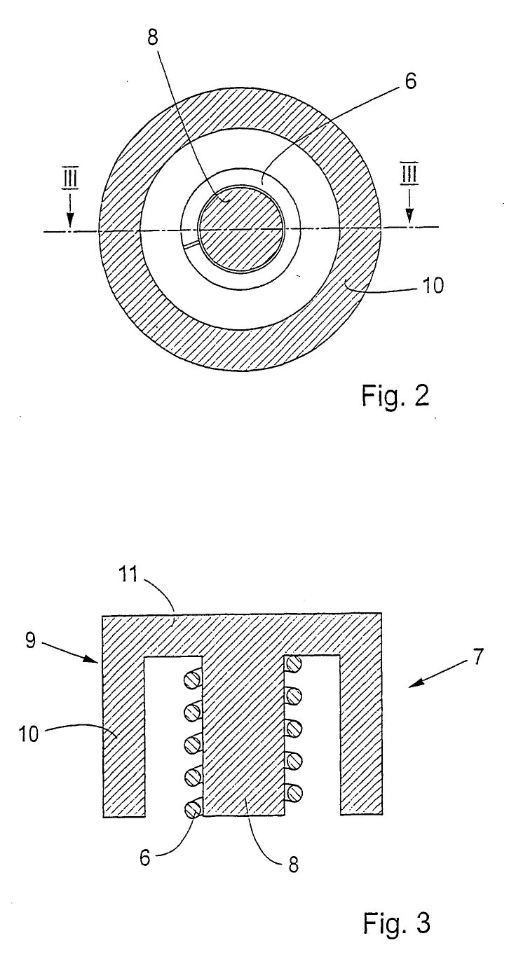

FIG. 2 is a front view of a coil and shield of a hand-held transmission device according to an embodiment of the invention,

FIG. 3 is a cross-section along the line III-III in FIG. 2,

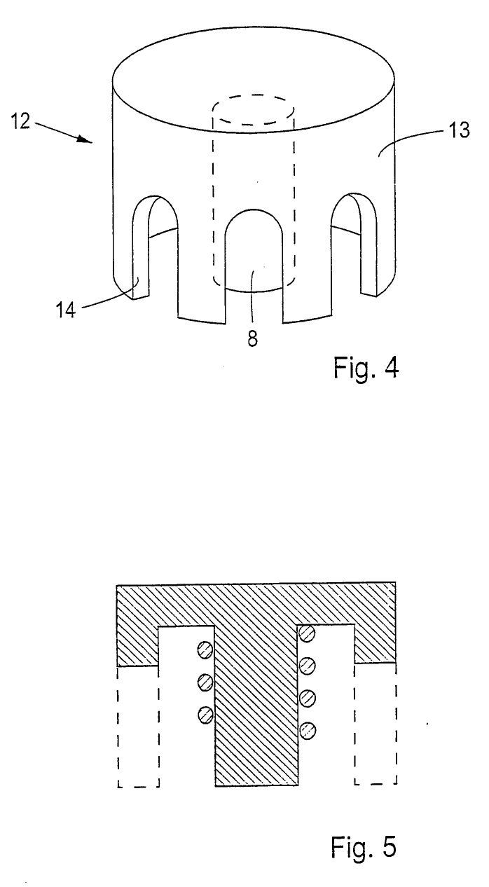

FIG. 4 is a perspective view of a coil and shield of a hand-held transmission device according to another embodiment of the invention,

FIG. 5 is a cross-section through the shield shown in FIG. 4,

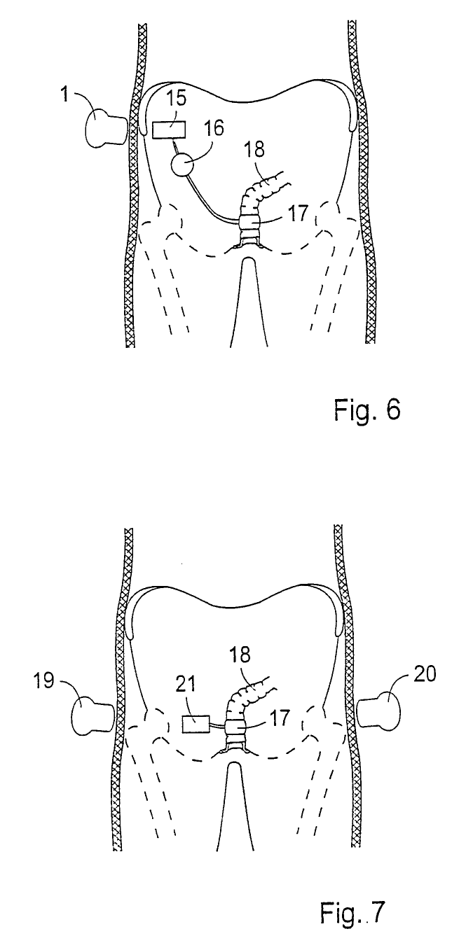

FIG. 6 illustrates an embodiment of the apparatus of the invention used for transferring energy to an artificial sphincter applied on the rectum of an anal incontinent human being, and

FIG. 7 is a modification of the embodiment according to FIG. 6.

FIG. 1 illustrates manual operation of an apparatus of the invention including an alternating magnetic field transmission device 1 held by an operator's hand 2 and a receiver 3 subcutaneously implanted in a human's body 4. The implanted receiver 3 is capable of receiving the alternating magnetic field transmitted by the transmission device 1 and of drawing energy from the alternating magnetic field to be supplied to an energy consuming medical device implanted in the body 4. The transmission device 1 includes a plastic box 5 containing a coil 6 adapted to generate an alternating magnetic field in the direction away from the hand 2 towards the implanted receiver 3. The coil 6 is situated in the lower part of the box 5 a few centimeters from the hand 2.

FIGS. 2 and 3 show a shield 7 of the apparatus for shielding the hand 2 from the alternating magnetic field including a ferrite core 8 extending in the coil 6 along the entire longitudinal extension thereof. (Alternatively, the coil 6 may be shorter than the core 8.) A ferrite casing 9 is integrated with the core 8 and surrounds the top rear end of the coil 6 and the circumference of the coil 6 along the entire longitudinal extension of the coil 6. The casing 9 includes a circular cylindrical wall 10 surrounding the coil 6 and a circular gable wall 11 joined to the cylindrical wall 10.

In FIG. 1 the shield 7 has a somewhat modified design, the position of the shield 7 being indicated in dotted lines. Thus, the cylindrical wall 10 extends only halfway along the coil 6.

FIGS. 4 and 5 show a modified shield 12 that does not dampen the alternating magnetic field as much as the shield 7 of FIGS. 2 and 3. Thus, the cylindrical wall 13 of the shield 12 is provided with several cutouts 14 evenly distributed around the circumference of the cylindrical wall 13. In this embodiment, the coil 6 is shorter than the core 8.

FIG. 6 shows an embodiment of the apparatus used for an anal incontinent human. The apparatus includes the transmission device 1 and a receiver 15 subcutaneously implanted in the human's body. The receiver 15 supplies energy to an implanted operation device 16 that operates an artificial sphincter 17 applied on the human's rectum 18. The transmission device 1 is held by an operator who puts it on the human's skin substantially in front of the implanted receiver 15 to provide efficient energy transmission from the transmission device 1 to the receiver 15.

The embodiment shown in FIG. 7 is identical to the embodiment shown in FIG. 6, except that the transmission device 1 include two wireless energy transmitters 19 and 20, and the receiver and operation device are integrated in a single receiver/operation unit 21 located close to the artificial sphincter 17. Each transmitter 19,20 may be designed as the transmission device 1 described above in connection with FIGS. 1-5. In this embodiment the transmitters 19 and 20 are positioned at different sides of the implanted receiver/operation unit 21, suitably at opposite sides as illustrated in FIG. 7, to transmit wireless energy to the receiver/operation unit 21.

Of course, two wireless energy transmitters may also be used for transmitting energy to the receiver 15 of the embodiment shown in FIG. 6.

1. A transmission device (1; 19,20) for transmitting an alternating magnetic field to a receiver (3; 15;21), which is implanted in a human's or animal's body to supply energy drawn from the alternating magnetic field to an energy consuming implant (17) in the human's or animal's body, the transmission device comprising a coil (6) adapted to generate the alternating magnetic field in a desired direction towards the implanted receiver, the coil having a longitudinal extension, a front end to be directed towards the receiver and a rear end to be directed away from the receiver, characterized by a shield (7; 12) adapted to shield the environment from the alternating magnetic field generated by the coil (6) except at the front end of the coil (6), the shield including a magnetizable core (8) extending in the coil and a magnetizable casing (9) integrated with the core and surrounding the rear end of the coil and the circumference of the coil along at least a portion of the longitudinal extension of the coil.

2. A transmission device according to claim 1, wherein the casing (9) completely surrounds the coil (6) except the front end thereof.

3. A transmission device according to claim 2, wherein the core (8) wholly extends along the longitudinal extension of the coil (6).

4. A transmission device according to claim 1, wherein the casing (9) surrounds the circumference of the coil (6) along a portion of the longitudinal extension of the coil.

5. A transmission device according to claim 4, wherein the core (8) and/or coil (6) extends past the casing (9) along the longitudinal extension of the coil, as seen in the direction towards the front end of the coil.

6. A transmission device according to claim 1, wherein the casing (9) comprises a circular cylindrical wall (10; 13) and a circular gable wall (11) joined to the cylindrical wall, the core (8) extends centrally in the cylindrical wall from the gable wall and the coil (6) is applied on the core with the rear end of the coil facing the gable wall.

7. A transmission device according to claim 6, wherein the cylindrical wall (13) is provided with cut-outs (14).

8. A transmission device according to claim 1, wherein the shield (7; 12) is made of ferrite.

9. A transmission device according to claim 1, further comprising a plastic box (5), in which the coil (6) and shield (7; 12) are arranged such that they are located at a distance, in the order of centimeters, from an operator's hand (2), when the operator holds the transmission device during operation.

10. An apparatus for wireless transfer of energy from outside a human's or animal's body to an energy consuming medical device (17) implanted in the human's or animal's body, comprising a transmission device (1; 19,20) operable from outside the human's or animal's body for transmission of an alternating magnetic field, and a receiver (3; 15; 21) implantable in the human's or animal's body for receiving and drawing energy from the alternating magnetic field to be supplied to the energy consuming implanted medical device, the transmission device including at least one coil (6) for generating the alternating magnetic field in a desired direction towards the receiver, the coil having a longitudinal extension, a front end to be directed towards the receiver and a rear to be directed away from the receiver, characterized by at least one shield (7) adapted to shield the environment from the alternating magnetic field generated by the coil (6) except at the front end of the coil (6), the shield including a magnetizable core (8) extending in the coil and a magnetizable casing (9) integrated with the core and surrounding the rear end of the coil and the circumference of the coil along at least a portion of the longitudinal extension of the coil.

11. An apparatus according to claim 10, wherein the transmission device comprises a first transmitter (19) and a second transmitter (20), the first and second transmitters include first and second coils (6), respectively, for generating respective alternating magnetic fields in two different directions towards the receiver (21), each of the first and second coils having a longitudinal extension, a front end to be directed towards the receiver and a rear end to be directed away from the receiver, and the first and second transmitters include first and second shields (9) adapted to shield the environment from the alternating magnetic fields, each of the first and second shields including a magnetizable core (8) extending in the coil and a magnetizable casing (9) integrated with the core and surrounding the rear end of the coil and the circumference of the coil along at least a portion of the longitudinal extension of the coil.

12. An apparatus according to claim 10, wherein the casing (9) completely surrounds the coil (6) except the front end thereof.

13. An apparatus according to claim 12, wherein the core (8) wholly extends along the longitudinal extension of the coil (6).

14. An apparatus according to claim 10, wherein the casing (9) surrounds the circumference of the coil (6) along a portion of the longitudinal extension of the coil.

15. An apparatus according to claim 14, wherein the core (8) and/or coil (6) extends past the casing (9) along the longitudinal extension of the coil, as seen in the direction towards the front end of the coil.

16. An apparatus according to claim 10, wherein the casing (9) comprises a circular cylindrical wall (10; 13) and a circular gable wall (11) joined to the cylindrical wall, the core (8) extends centrally in the cylindrical wall from the gable wall and the coil is applied on the core with the rear end of the coil facing the gable wall.

17. An apparatus according to claim 16, wherein the cylindrical wall (13) is provided with cut-outs (14).

18. An apparatus according to claim 10, wherein the shield (7) is made of ferrite.

19. An apparatus according to claim 10, further comprising a plastic box (5), in which the coil (6) and shield (7) are arranged such that they are located at a distance, in the order of centimeters, from an operator's hand, when the operator holds the transmission device (1; 19,20) during operation.

20. A method for harmless wireless transfer of energy to an energy consuming medical device (17) implanted in a human's or animal's body, comprising implanting in the human or animal a receiver (3; 15; 21) capable of receiving and drawing energy from an alternating magnetic field to be supplied to the energy consuming medical device, manually holding external to the body a transmission device (1; 19,20) capable of transmitting the alternating magnetic field, the transmission device including a coil (6) for generating the magnetic field and having a longitudinal extension, a front end directed away from the hand holding the transmission device and a rear end facing the hand holding the transmission device, and transmitting by means of the transmission device the alternating magnetic field to the implanted receiver, characterized by shielding by means of a shield (7) the hand holding the transmission device (1; 19,20) from the alternating magnetic field generated by the coil (6), the shield including a magnetizable core (8) extending in the coil (6) of the transmission device and a magnetizable casing (9) integrated with the core of the transmission device and surrounding the rear end of the coil and the circumference of the coil along at least a portion of the longitudinal extension of the coil.

21. A method for harmless wireless transfer of energy to an energy consuming medical device (17) implanted in a human's or animal's body, comprising implanting in the human or animal a receiver (3; 15; 21) capable of receiving and drawing energy from an alternating magnetic field to be supplied to the energy consuming medical device, providing an external transmission device (1; 19,20) capable of transmitting the alternating magnetic field and including a coil (6) having a longitudinal extension, a front end and a rear end, positioning the transmission device relative to the body so that the front end of the coil is directed towards the receiver and the rear end of the coil is directed away from the receiver, and transmitting by means of the transmission device the alternating magnetic field to the implanted receiver, characterized by shielding by means of a shield (7) the environment from the alternating magnetic field generated by the coil (6) except at the front end of the coil (6), the shield including a magnetizable core (8) extending in the coil of the transmission device and a magnetizable casing integrated with the core of the transmission device and surrounding the rear end of the coil and the circumference of the coil along at least a portion of the longitudinal extension of the coil.
